(19) United States
(12) Plant Patent Application Publication
(10) Pub. No.: US 2003/0037360 P1
(43) Pub. Date: Feb. 20, 2003

(54) Ilex hybrid variety named 'Dapat'

(76) Inventor: David Taylor, Semmes, AL (US)

Correspondence Address: David W. Taylor, P.O. Box 144, Semmes, AL, 36575, US

(21) Appl. No.: 09/919,572

(22) Filed: Aug. 1, 2001

(51) Int. Cl. A01H 5/00

(52) U.S. Cl. PLT/247

(57) ABSTRACT

A new and distinct variety of evergreen Ilex plant selected from a controlled cross of Ilex hybrid ‘Mary Nell’ and Ilex hybrid aquipemyi. This new variety has great merit as a commercially produced landscape plant due to its unique combination of parental cold hardiness characteristics and small dense foliage.

### CROSS-REFERENCE TO RELATED APPLICATIONS

| | | | |
|---|---|---|---|
| PP8,792 | June 1994 | Fischer | Plt/65 |
| PP9,484 | March 1996 | Magee | Plt/65 |
| PP9,485 | March 1996 | Magee | Plt/65 |

### STATEMENT AS TO RIGHT TO INVENTION MADE UNDER FEDERALLY-SPONSORED RESEARCH AND DEVELOPMENT

None

### BACKROUND OF THE INVENTION 1. Field of the Invention

This invention relates to a new variety of female, evergreen, holly plant. This plant, Ilex hybrid ‘Dapat’ (Ilex ‘Mary Nell’×(Ilex ×aquipemyi)), is unique to my research and experience. This new and distinct variety is herein shown and described, introducing its merits as a vigorous, upright grower with dense small leaves and suggesting its commercial uses as hedges or specimens. This new variety will be sold under the trademarked name Miss Patricia.

2. Description of relevant prior art including information disclosed under 3 7 CFR 1.97-1.99

This new variety of Ilex holly was selected from seedlings planted at Taylor's Nursery in Semmes, Ala. The seedlings were a result of a controlled hybridization cross I made using the female holly Ilex hybrid ‘Mary Nell’ and a male holly specimen of Ilex hybrid aquipernyi. This cross was made in 1990 and the seedling was selected in 1992. I have closely guarded this holly while I evaluated it at Taylor's Nursery. I have been very successful in asexual propagation of this plant at my nursery, and it has proven to be a clonable, stable form of holly through several generations. The female parent was chosen due to its green glossy foliage and the perceived need to improve on foliage density and leaf size. The male parent was chosen simply to improve the cold hardiness, as the male's parents listed to Zone 5b and 6b.

### SUMMARY OF THE INVENTION

I have listed the following as the distinguishing characteristics of this new holly variety, the combination of all being a unique and new plant.

1. Dense, small foliage.

2. Upright and naturally pyramidal.

3. Heat and drought tolerant.

4. Commercially accepted horticulture practices produce saleable vigorous plants easily.

5. Not stunted by heavy shearing.

6. Can be used for topiary—poodle form—products.

### BRIEF DESCRIPTION IF THE DRAWING

The colors shown are as true as is reasonably possible to obtain by the means at my disposal. The colors are defined with reference to The Royal Horticultural Society Color Chart.

FIG. 1 reveals the mature foliage characteristics of ‘Dapat’ as compared to three other hollies in the same botanical and market class.

FIG. 2 discloses the leaf characteristics of ‘Dapat’.

FIG. 3 shows a group of the ‘Dapat’ clones.

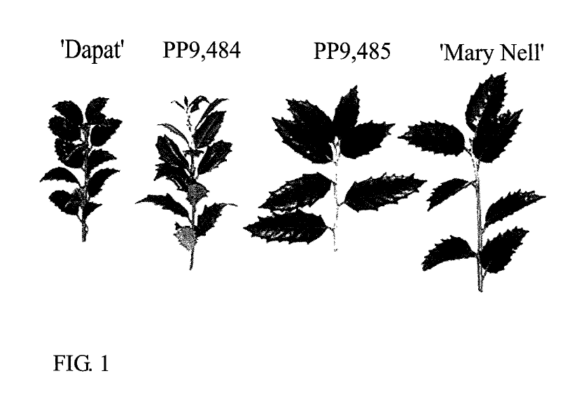

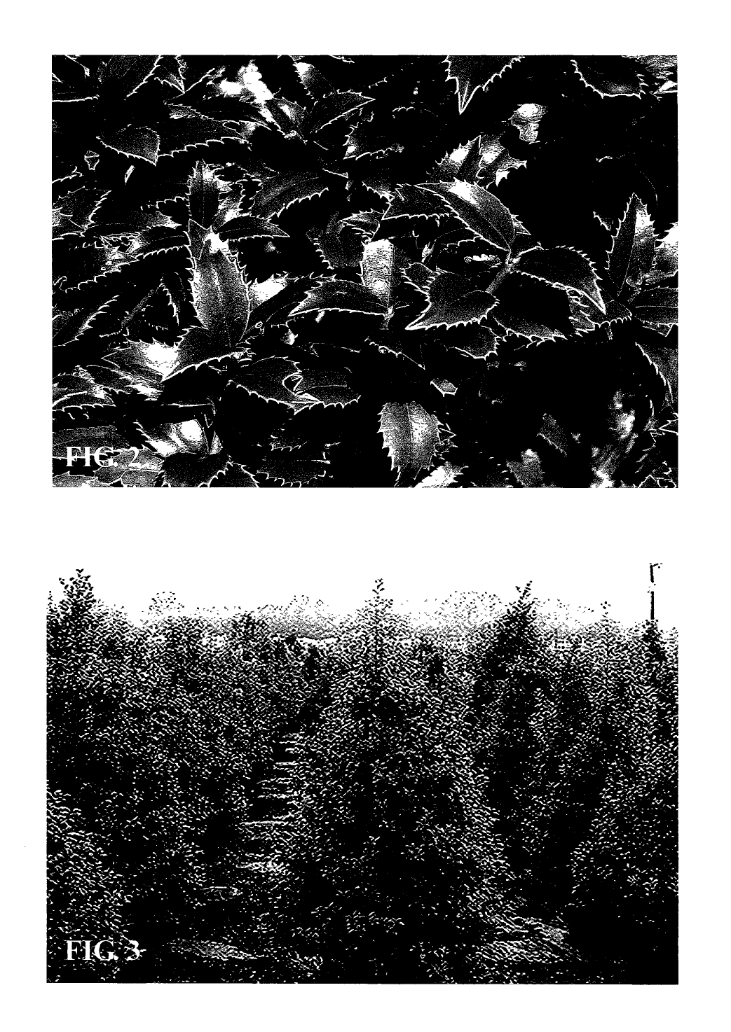

### BOTANICAL DESCRUPTION OF THE PLANT

I will compare my plant to three others in the same botanical and market class.

Distinctive Characteristics:

| | | PP9,484 | PP9,485 | |
|---|---|---|---|---|
| Characteristic | ‘Dapat’ | ‘Coned’ | ‘Conal’ | ‘Mary Nell’ |
| Mature Height | 8′-10′ | 10′-12′ | 15′-20′ | 15′-20′ |
| Mature Width | 4′-5′ | 8′-10′ | 12′-15′ | 12′-15′ |
| Leaf Length | 1⅜″-1 9/16″ | 2″-2¾″ | 2⅜″-3¾″ | 2⅝″-3½″ |
| Leaf Width | 11/16″-⅞″ | ⅞″-1¼″ | 1⅛″-1½″ | 1⅛″-1¾″ |
| Mature Leaf | Closest to | Closest to | Closest to | Green Group |
| Color | Green Group | Green Group | Green Group | 139A |
| | 139A | 139A | 139A | |
| Immature Leaf | Closest to | Grayed | Grayed | Green Group |
| Color | Yellow | Purple | Purple | 144A |
| | Green Group | Group 187A | Group 187A | |
| | 144A | | | |
| Mature Leaf | Ovate | Ovate to | Ovate to | Ovate to |
| Shape | | Narrowly | Lanceolate | Broadly |
| | | Lanceolate | | Lanceolate |
| Leaf Base | Ovate to | Cuneate | Obtuse | Obtuse |
| | Slightly | | | |
| | Cuneate | | | |
| Pairs | 11-13 | 7-9 | 9-11 | 9-11 |
| Leaf Spines | | | | |
| Flower Sex | Female Only | Female Only | Female Only | Female Only |
| Mature Shape | Upright | Upright | Upright | Upright |
| | Dense | Dense | Pyramidal | Pyramidal |
| | Pyramidal | Global | | |
| Cold Hardiness | 7a | 7 | 7 | 7 |
| Mature Fruit | Orange-Red | Orange-Red | Red Group | Red Group |
| Color | Group 33A | Group 33A | 53A | 40A |

Classification:

Botanic.—Ilex Hybrid ‘Dapat’.

Form.—Upright, Dense, Pyramidal.

Texture.—Medium.

Height.—8′-10′.

Width.—4′-5′.

Growth habit: Upright, dense, pyramidal. Vigorous upright growth with superior self-branching characteristics. Fast growth rate.

Foiage: Alternate, simple, evergreen, ovate, and vary in size from 1 3/8″-1 8/16″ long and 11/16″-7/8″ wide. The margins are serrate to doubly serrate with 11-13 pairs of spines. The spines vary in length from 5/64″ to 5/32″. The leaf apex is acute to slightly acuminate. The mature leaf color is closest to green group 139A. The immature leaf color is closest to yellow-green group 144A.

Stems: New growth is closest to yellow-green group 144B Older stems are closest to green group 137B After the stems mature they are closest to gray-brown group 199C

Flower: Small whitish-yellow, inconspicuous, borne on previous season's growth. The flowers are typical of female holly, pistillate small and borne in cluster. Bloom time is generally typical of the evergreen species in the market class, in my nursery on the Alabama Gulf Coast—early to mid April.

Fruit: Globose, 1/4″-3/8″ diameter. The mature color is closest to Orange-Red Group 33A. Fruit set is variable with moderate crops being the norm, as compared to other hollies in the same botanical and market class.

Culture: This holly grows very well in a wide range of environments. Full sun, with no scorching to partial shade. Grows in nearly any soil type, from moist to dry and sandy clay. The original unattended specimen has thrived through official drought and several extended deluges associated with named Hurricanes.

I claim:

1. A very distinct, unique new variety of evergreen Ilex holly plant named Ilex hybrid ‘Dapat’ as described and illustrated herein.
